(19) United States
(12) Patent Application Publication
(10) Pub. No.: US 2021/0386506 A1
(43) Pub. Date: Dec. 16, 2021

(54) MEDICAL CHAIR PROTECTIVE BARRIER AND GAS DISTRIBUTION SYSTEM

(71) Applicant: Jason Gee, Arcadia, CA (US)

(72) Inventor: Jason Gee, Arcadia, CA (US)

(21) Appl. No.: 16/902,211

(22) Filed: Jun. 15, 2020

(51) Int. Cl. A61B 90/40; A61G 15/14; A61G 15/10

(52) U.S. Cl. CPC A61B 90/40; A61B 2090/401; A61G 15/10; A61G 15/14

(57) ABSTRACT

A medical chair protective barrier and gas distribution system provides a frame that forms a sealed environment for a medical gas delivered to head of a dental chair. The frame fastens to the medical chair, or rolls to head of medical chair. A laminar flow subassembly secures to frame. The laminar flow subassembly includes a vacuum pump, tubing, positive and negative pressure ports, and a vent for delivering medical gas to the head of medical chair. The frame carries a hood that covers the head of medical chair. The hood retains medical gases in a sealed environment, while protecting dentist from contact with medical gases. The hood includes a window and a pair of arm passages that regulates access to inside of hood. Medical armamentarium hangs on frame for access. System has inlet and outlet vacuum ports that utilize HEPA filters and hidden UV lights for disinfecting under the hood.

### FIELD OF THE INVENTION

The present invention relates generally to a medical chair protective barrier and gas distribution system. More so, the present invention relates to a protective barrier and gas distribution system that is operable with a medical chair, provides a mobile frame that operates adjacent to the head area of a medical chair to: deliver a medical gas to the head area of the medical chair, support a protective hood for containing the medical gas in the head area and preventing the medical professional from contacting the medical gas, vent the medical gas out of the protective hood through an exhaust port, and retain at least one medical armamentarium within easy access to the medical professional.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, a reclining chair used in the field of dentistry is an elongated chair or bed, that is adjustable in height and is shaped to support the patient's body, generally from the head to the lower legs. The chair can usually be reclined to position the patient, and particularly the patient's mouth, for convenience to the dentist and dental technician.

Dental interventions in patients which are performed in dental offices require operating with a high degree of local sterility, in order to ensure health and safety for the patient. In particular, in dental medical offices, a high degree of sterility can be desired in an extremely reduced operative area. Such need does not typically justify the extremely burdensome use of certain methods and apparatuses typically employed in the hospital operating rooms. Thus, an aerosol that is produced from dental instruments like ultrasonic scalers, dental handpieces, three-way syringes and other high-speed instruments. These aerosols are air suspended in the clinical environment. These aerosols can pose risks to the clinical, staff and other patients as well.

Other proposals have involved dental chair sterilization systems. The problem with these dental systems is that they do not efficiently deliver aerosols to the head of the patient. Also, the dentist is not protected from splatter and contamination from the aerosol. Even though the above cited dental systems meet some of the needs of the market, a protective barrier and gas distribution system that is operable with a medical chair, provides a mobile frame that operates adjacent to the head area of a medical chair to: deliver a medical gas to the head area of the medical chair, support a protective hood for containing the medical gas in the head area and preventing the medical professional from contacting the medical gas, vent the medical gas out of the protective hood through an exhaust port, and retain at least one medical armamentarium within easy access to the medical professional, is still desired.

### SUMMARY

Illustrative embodiments of the disclosure are generally directed to a medical chair protective barrier and gas distribution system. The medical chair protective barrier and gas distribution system serves to create a protective environment near the head of a medical chair that introduces, contains, and vents a medical gas at the head area of the patient.

In some embodiments, the medical chair protective barrier and gas distribution system provides a frame that forms a protective environment near the head of a medical chair. A laminar flow subassembly secures to the frame. The laminar flow subassembly includes a vacuum pump, tubing, positive pressure inlet ports, negative pressure outlet ports, and a vent for delivering medical gas to the head area of the medical chair; and consequently, to the head of the patient. The frame carries a protective hood that covers the head of area of the medical chair. The hood retains the medical gas in a sealed environment, while also allowing the medical professional access to the patient. The hood is resilient, opaque, and thereby, configured to substantially contain the medical gas in proximity the head area while protecting the medical professional from contact with the medical gas. A window in the hood regulates access to the inside of hood. Additionally, the frame helps retain at least one medical armamentarium to enable easy access by the medical professional.

In essence, the invention provides a net negative pressure hood or canopy for a dental delivery unit. The whole concept of an all in one dental delivery unit that can help mitigate aerosol, contagion spread, and even self-disinfect, while increasing dentist efficiency as well as patient comfort.

In one aspect, a medical chair protective barrier and gas distribution system, comprises:
a frame comprising at least two vertical bars interconnecting at least two horizontal bars, the bars being pivotable about a hinge;
a laminar flow subassembly supported by the horizontal bars, the laminar flow subassembly comprising one or more inlet ports, at least one tube, one or more outlet ports, and a vent, the laminar flow subassembly operable to enable flowage of a fluid from the inlet ports, through the tubing, and to the outlet ports;
a vacuum subassembly operatively connected to the laminar flow subassembly, the vacuum subassembly operable to displace the fluid from the inlet ports to the outlet ports; and
a hood substantially covering the frame, the hood being operable to substantially prevent escape of the fluid from the approximate area of the frame

In another aspect, the frame is operable in proximity to a head region of a medical chair.

In another aspect, the frame fastens to the medical chair.

In another aspect, the frame rolls to the head region of the medical chair.

In another aspect, the medical chair comprises a dental chair.

In another aspect, the at least two vertical bars comprise four vertical bars.

In another aspect, the system further comprises a mobility mechanism at a bottom end of the vertical bars.

In another aspect, the mobility mechanism includes at least one of the following: a wheel, a castor wheel, a roller, a blade, and a slick mat.

In another aspect, the at least two horizontal bars comprise a U-shaped arrangement of horizontal bars supporting the vertical bars, the horizontal bars further comprising multiple transverse support bars.

In another aspect, the fluid comprises a gas.

In another aspect, the gas comprises a dental aerosol.

In another aspect, the vacuum subassembly comprises a vacuum pump, a positive pressure port, and a negative pressure port, the vacuum pump being operable to displace the fluid from the positive pressure port to the negative pressure port.

In another aspect, the vent of the laminar flow subassembly comprises an FDA approved HVAC subassembly, or a filtration subassembly, or both.

In another aspect, the hood is resilient.

In another aspect, the hood is at least partially transparent.

In another aspect, the hood comprises a window, the window enabling access to the area in proximity to the frame.

In another aspect, the hood is fabricated from a clear acrylic material.

In another aspect, the medical gas comprises infectious aerosols, disinfectants, and air.

One objective of the present invention is to prevent escape of dental aerosols from the head region of a dental chair with a protective hood.

Another objective is to minimize compromise to patient comfort and the medical professional in the course of performing dental procedures.

Yet another objective is to retrofit the frame, laminar flow subassembly, and hood to existing dental chairs and delivery systems.

An exemplary objective is to prevent greater than 90% of gas and aerosol from escaping; and also prevent 100% of splatter from transferring from the patient to the medical professional.

Yet another objective is to mitigating transfer of infectious agents from the patient area to the medical professional.

Another objective is to provide a disposable frame.

Additional objectives are to provide an inexpensive to manufacture medical chair protective barrier and gas distribution system.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a perspective view of an exemplary medical chair protective barrier and gas distribution system, in accordance with an embodiment of the present invention;

FIG. 2 illustrates a perspective view of an exemplary frame for the medical chair protective barrier and gas distribution system, in accordance with an embodiment of the present invention;

FIG. 3 illustrates a perspective view of an exemplary laminar flow subassembly, showing the inlet ports, in accordance with an embodiment of the present invention;

FIG. 4 illustrates a frontal perspective view of an exemplary hood for the medical chair protective barrier and gas distribution system, in accordance with an embodiment of the present invention;

FIG. 5 illustrates a side view of the hood, in accordance with an embodiment of the present invention;

FIG. 6 illustrates a top perspective view of the hood, in accordance with an embodiment of the present invention;

FIG. 7 illustrates a perspective view of a medical professional accessing a patient through a window and a pair of arm passageways that form in the hood, in accordance with an embodiment of the present invention;

FIG. 8 illustrates a top view of the medical chair protective barrier and gas distribution system, showing a patient connected to air tubes, in accordance with an embodiment of the present invention;

FIG. 9 illustrates a side view of the medical chair protective barrier and gas distribution system, showing a patient connected to air tubes, in accordance with an embodiment of the present invention; and

FIG. 10 illustrates a side view of the medical chair protective barrier and gas distribution system, showing a plexiglass bubble visor styled hood, in accordance with an embodiment of the present invention.

Like reference numerals refer to like parts throughout the various views of the drawings.

### DETAILED DESCRIPTION OF THE INVENTION

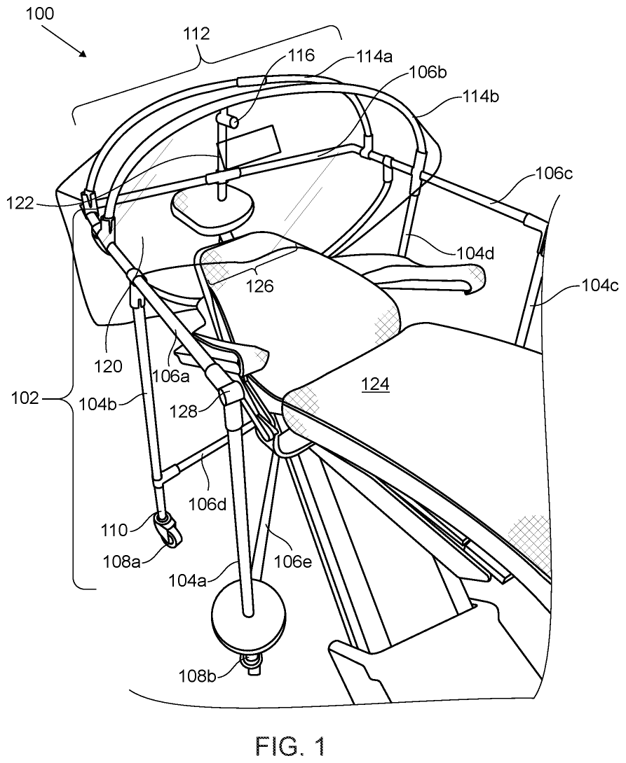

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A medical chair protective barrier and gas distribution system 100 is referenced in FIGS. 1-10. The medical chair protective barrier and gas distribution system 100, hereafter “system 100” provides a unique medical tool that retrofits, or slidably moves adjacent to a medical chair 124 to introduce and retain a fluid 304 near a head region 126 of a medical chair 124, while also preventing a medical professional from receiving splatter or being covered with the fluid 304.

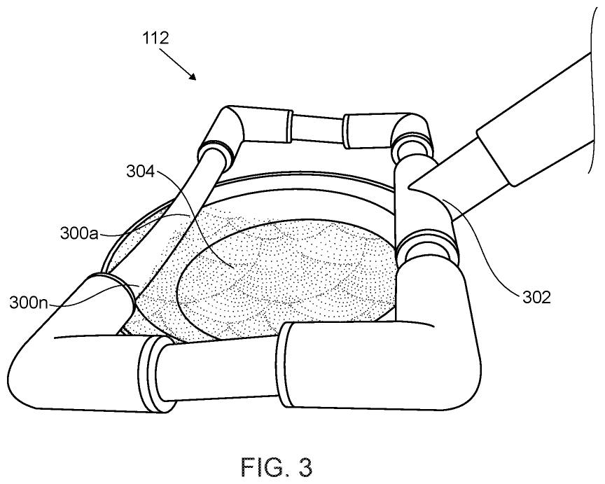

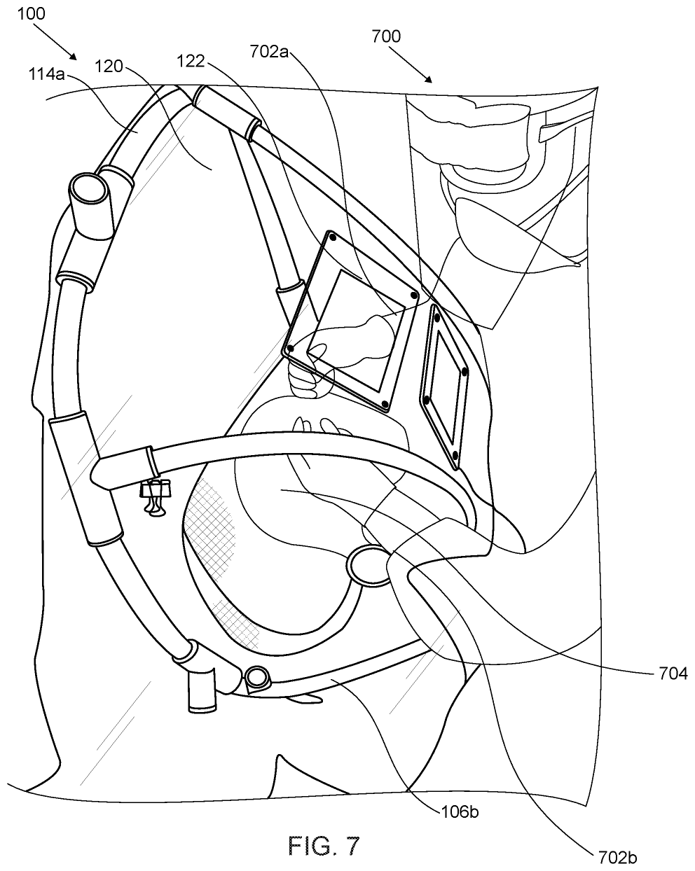

As FIG. 1 shows, the medical chair 124 may include a dental chair having a head region 126 oriented to where the head of a patient rests. In one example, the medical chair 124 is shaped to support the patient's body, generally from the head to the lower legs. The chair can usually be reclined to position the patient, and particularly the patient's mouth, for convenience and safety to a medical professional 700 (See FIG. 7), which may include a dentist and a dental technician. The fluid 304 (See FIG. 3) may include a medical gas, such as an aerosol or an anxiolytic gas, known in the field of dentistry. The medical gas may also include, without limitation, infectious aerosols, disinfectants, and air.

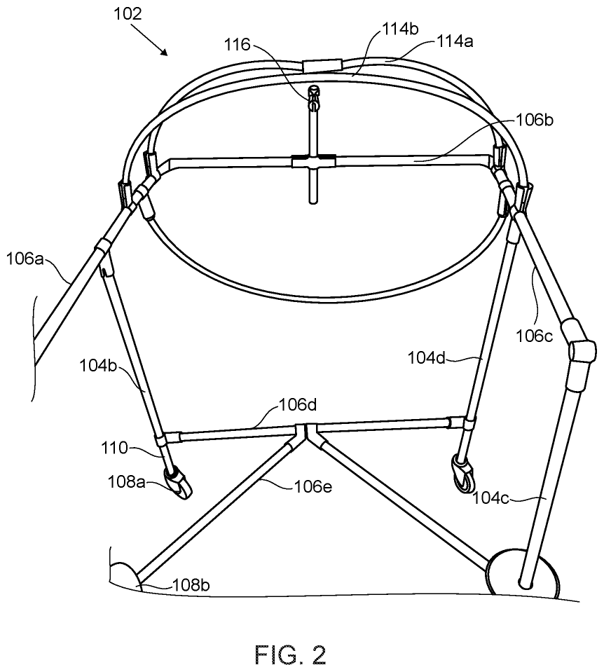

Looking now at FIG. 2, the system 100 comprises a frame 102 that forms the primary support structure for the system 100. In some embodiments, the frame 102 is operable in proximity to a head region 126 of the medical chair 124, so as to introduce the fluid 304 at or near the head of a patient. This is illustrated in FIG. 1. In some embodiments, the frame 102 fastens to the medical chair 124. In other embodiments, the frame 102 rolls/slides over the head region 126 of the medical chair 124. In any case, the system is operable with the medical chair 124. In one possible embodiment, the bars terminate at a skirt. The skirt forms port holes for passage of the bars. The skirt provides yet another protective shield for the medical professional and the patient.

In some embodiments, the frame 102 may comprise of at least two vertical bars 104a-d that interconnect with at least two horizontal bars 106a-e. In one non-limiting embodiment, the at least two vertical bars 104a-d comprise four vertical bars 104a-d. In other embodiments, the at least two horizontal bars 106a-e comprise a U-shaped arrangement of horizontal bars 106a-e supporting the vertical bars 104a-d. The horizontal bars 106a-e further comprising multiple transverse support bars that add structural integrity to the frame 102. In other embodiments, the bars 104a-d, 106a-e are pivotable about a hinge 128. However, the bars may also be stationary. Suitable materials for the frame 102 may include, without limitation, steel, aluminum, metal alloy, a rigid polymer, and wood.

In some embodiments, the system 100 may also utilize a mobility mechanism 108a-b at a bottom end 110 of the vertical bars 104a-d. The mobility mechanism 108a, 108b may include, without limitation, a wheel, a castor wheel, a roller, a blade, and a slick mat. Thus, the mobility mechanism 108a-b allows the frame 102 to roll or slide in proximity to the head region 124 of the medical chair 126. Additionally, the frame 102 helps retain at least one medical armamentarium to enable easy access by the medical professional 700. For example, the horizontal bars serve as hanging surfaces for dental instruments.

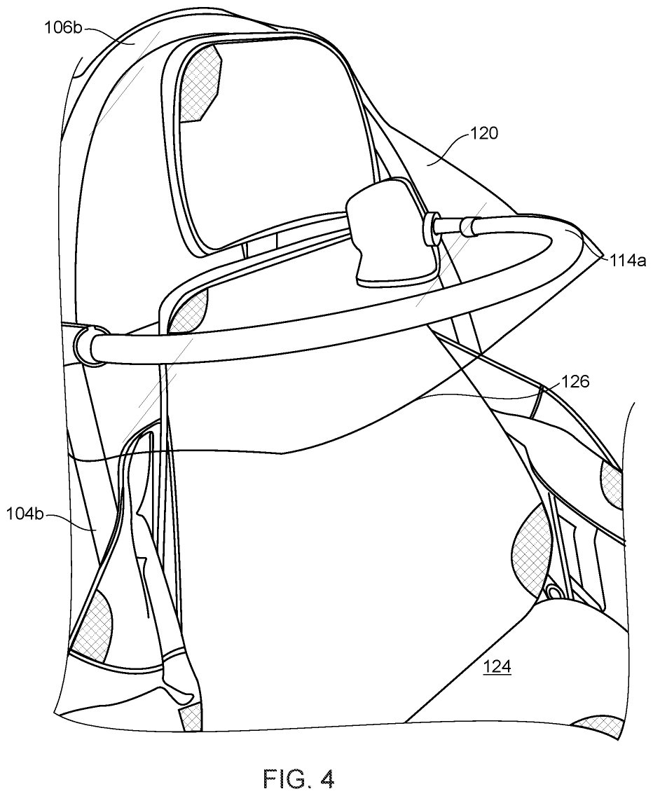

As illustrated in FIG. 1, a laminar flow subassembly 112 introduces the fluid to the medical chair. The laminar flow subassembly 112 is supported by the horizontal bars 106a-e and/or the vertical bars 104a-d. In one possible embodiment, the laminar flow subassembly 112 includes one or more inlet ports 300a, 300n that serve to introduce the fluid 304. The inlet ports 300a-n are shown in FIG. 3. The laminar flow subassembly 112 also includes at least one tube 114a, 114b for carrying the fluid 304; one or more outlet ports 118 for discharging the fluid 304 near the patient's head; and a vent 116 for exhausting the fluid 304 from beneath the hood 120 (Note, hood 120 is shown in FIG. 4). Thus, the laminar flow subassembly 112 is configured to enable flowage of the fluid 304 (See FIG. 3) from the inlet ports 300a, 300n, through the tube 114a-b, and finally to the outlet ports 116 above the head of the patient.

In one possible embodiment, the vacuum subassembly 112 has inlet ports 300a-n that are positive pressure ports. Conversely, the outlet ports 118 include a negative pressure port. As is known in the art, the fluid travels from the positive pressure to the negative pressure. This is especially true for gases. The laminar flow subassembly 112 may also include various types of internal electrical components, vacuum ports, compressed air, sand, and a water supply, as known in the art. These all serve to carry the fluid 304 to the head region 124 of the medical chair 126. In an alternative embodiment, the inlet and outlet vacuum ports 300a-n, 118 utilizes a high efficiency particulate air (HEPA) filter 802, and a hidden UV light 804 to provide additional disinfecting capacity.

In some embodiments, the system 100 utilizes a vacuum pump 302 that is configured to operatively connect to the laminar flow subassembly 112 (See FIG. 3). The vacuum pump 302 works to displace the fluid 304 from the inlet ports 300a, 300n, across the tube 114a-b, and finally to the outlet ports 116. Or in other terms, the vacuum pump 302 is operable to displace the fluid 304 from the positive pressure port to the negative pressure port. In some embodiments, the fluid 304 comprises a gas. In one non-limiting embodiment, the gas comprises a dental aerosol. The vacuum pump 302 may be powered by an internal battery or an external power source.

In one possible embodiment, the vent 116 of the laminar flow subassembly 112 comprises an FDA approved HVAC subassembly, and a filtration subassembly. It is known in the art that introducing a gas, such as an aerosol, to a dental patient is necessary when performing complicated dental procedures, including endodontics. Thus, the frame 102 brings the fluid 304 in proximity to the head region of the medical chair, the laminar flow subassembly 112 introduces the gas, and the hood 120 maintains the gas extensively in the confines of the head region 124 for the medical chair 126.

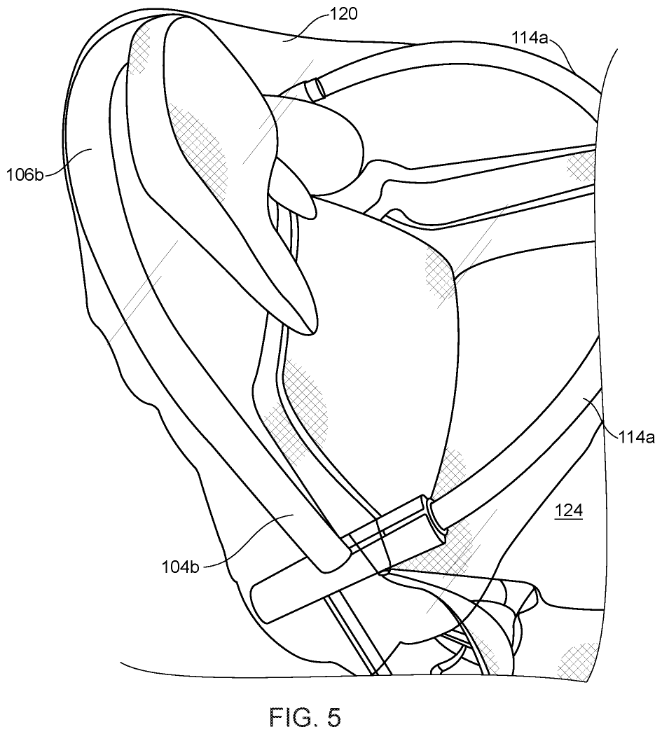

As illustrated in FIG. 4, the system 100 also includes a hood 120. The hood 120 can include a resilient fabric that drapes, or fastens across the frame 102, and possibly part of the laminar flow subassembly 112. The hood 120 is configured to substantially cover the frame 102. In some embodiments, the hood 120 is shaped and dimensioned to substantially prevent escape of the fluid from the approximate area of the frame 102. In essence the hood encapsulates the patient in a cabin that contains a medical gas, or additional anxiolytic functions for entertainment or distraction. In this manner, the hood 120 helps to prevent greater than 90% of gas and aerosol from escaping; and also prevent 100% of splatter from transferring from the patient to the medical professional 700. Thus, the hood 120 serves as a net negative pressure hood for a dental delivery unit. In some embodiments, the hood 120 is resilient and at least partially transparent (See FIG. 5). In one non-limiting embodiment, the hood is fabricated from a clear acrylic material.

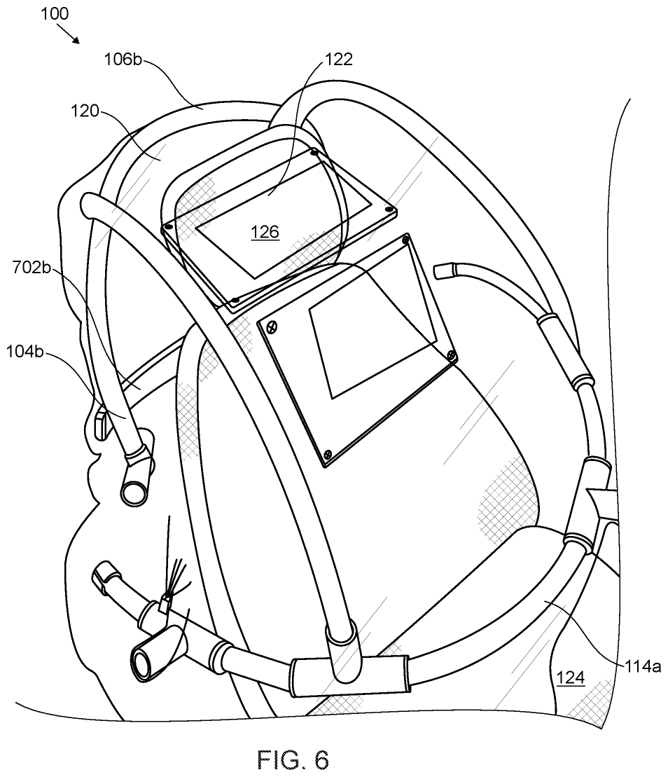

In one possible embodiment shown in FIG. 5, the hood 120 comprises a window 122. The window 122 is configured to open and close, so as to enable access to the area in proximity to the frame 102. For example, one possible hood 120 includes an acrylic clear plastic window that clamps over disposable plastic bags i.e., 60-gallon bags. The window sandwiches the plastic securely (See FIG. 6). In this manner, a window of opaque bag material can be safely removed without compromising seal of the “gasketed” plastic periphery. Further, the window 122 may be easily closed and opened for operating microscopes for endodontics. In additional embodiments of the hood, FIG. 10 illustrates the system, showing a plexiglass bubble visor styled hood 1000.

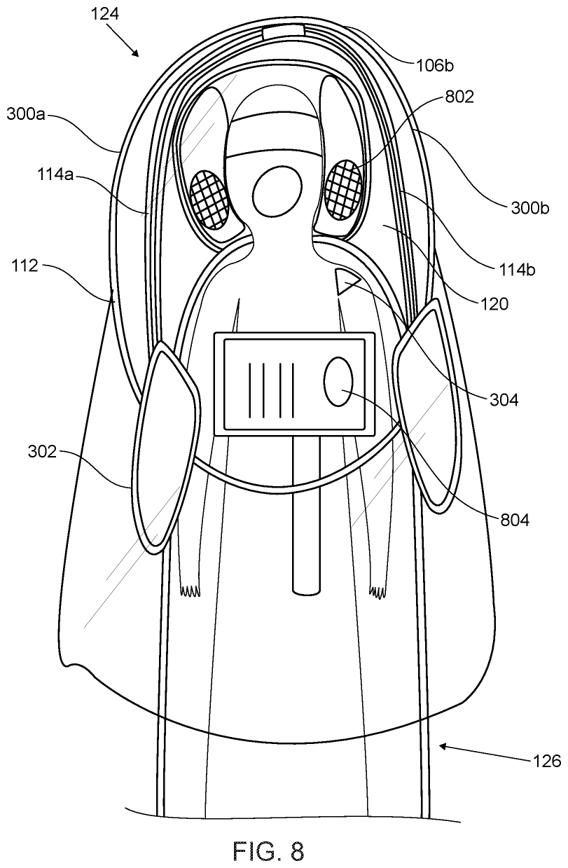

The hood 120 also forms a pair of arm passageways 702a, 702b at the lateral sides. The arm passageways 702a-b enable passage of the arms from the medical professional. The arm passageways 702a-b are sized and dimensioned to enable a snug fit for the arms, so that the medical professional can access the patient 704. FIG. 7 illustrates a perspective view of a medical professional accessing a patient through a window and a pair of arm passageways that form in the hood 120. FIG. 8 shows the patient lying in the medical chair.

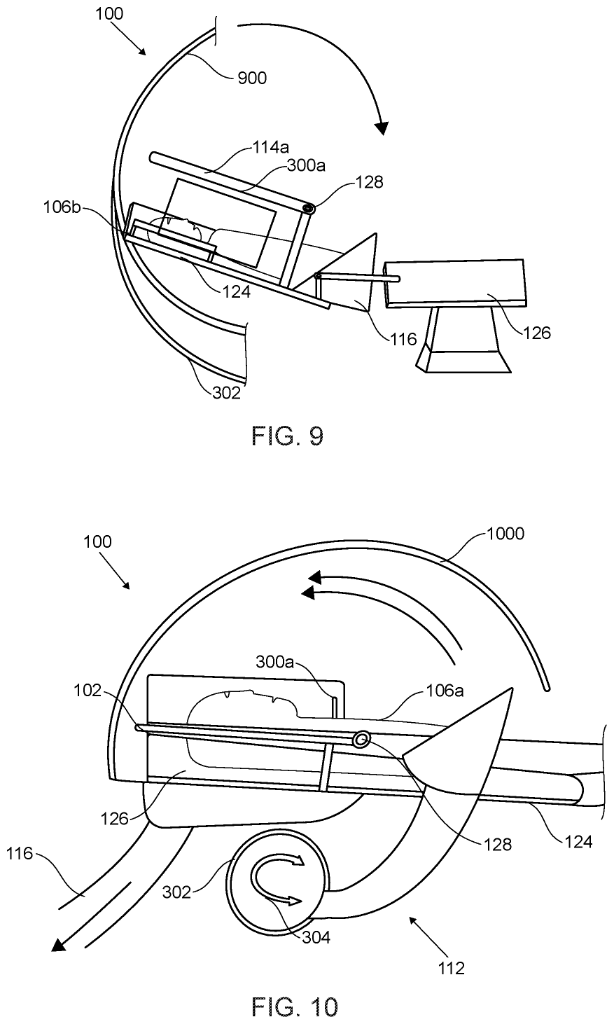

In conclusion, FIG. 9 illustrates a frame 102 that forms a protective, sealed environment for a medical gas delivered to the head of a dental chair. The frame 102 fastens to the medical chair 124, or rolls to the head of the chair. A laminar flow subassembly 112 secures to the frame 102. The laminar flow subassembly 112 may include a vacuum pump, tubing, positive pressure inlet ports, negative pressure outlet ports, and a vent. These components work to deliver a medical gas to the head region 124 of the medical chair 126; and consequently, to the head of a patient lying in the medical chair 126. As illustrated, a pivoting hood 900 can be configured to swing over the head of the patient.

Looking now at FIG. 10, the frame 102 carries a hood 120 across the head region 124 of the medical chair 126. In one non-limiting embodiment, the hood 120 is partially transparent, and configured to substantially retain the medical gas in a sealed environment, while also protecting medical professional 700 from contact with medical gases, aerosols, etc. The hood 120 includes a window 122 that regulates access to inside of hood 120. Medical armamentarium can hang on the frame 102 for easy access.

These and other advantages of the invention will be further understood and appreciated by those skilled in the art by reference to the following written specification, claims and appended drawings. Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A medical chair protective barrier and gas distribution system, the system comprising:
a frame comprising at least two vertical bars interconnecting at least two horizontal bars;
a laminar flow subassembly supported by the horizontal bars, the laminar flow subassembly comprising one or more inlet ports, at least one tube, one or more outlet ports, and a vent, the laminar flow subassembly operable to enable flowage of a fluid from the inlet ports, through the tubing, and to the outlet ports;
a vacuum pump operatively connected to the laminar flow subassembly, the vacuum subassembly operable to displace the fluid from the inlet ports to the outlet ports; and
a hood substantially covering the frame, the hood being operable to substantially prevent escape of the fluid from the approximate area of the frame.

2. The system of claim 1, wherein the frame is operable in proximity to a head region of a medical chair.

3. The system of claim 2, wherein the frame fastens to the medical chair.

4. The system of claim 3, wherein the frame rolls to the head region of the medical chair.

5. The system of claim 1, wherein the bars are pivotable about a hinge.

6. The system of claim 1, wherein the at least two vertical bars comprise four vertical bars.

7. The system of claim 1, further comprising a mobility mechanism disposed at a bottom end of the vertical bars.

8. The system of claim 7, wherein the mobility mechanism includes at least one of the following: a wheel, a castor wheel, a roller, a blade, and a slick mat.

9. The system of claim 1, wherein the at least two horizontal bars comprise a U-shaped arrangement of horizontal bars supporting the vertical bars, the horizontal bars further comprising multiple transverse support bars.

10. The system of claim 1, wherein the fluid comprises a gas.

11. The system of claim 1, wherein the inlet ports comprise a positive pressure port.

12. The system of claim 11, wherein the outlet ports comprise a negative pressure port.

13. The system of claim 12, wherein the vacuum pump is operable to displace the fluid from the positive pressure port to the negative pressure port.

14. The system of claim 1, wherein the hood forms a pair of arm passageways.

15. The system of claim 1, wherein the hood forms at least one window, the window enabling access to the area in proximity to the frame.

16. The system of claim 1, wherein the hood is resilient.

17. The system of claim 1, wherein the hood is fabricated from a clear acrylic material.

18. A medical chair protective barrier and gas distribution system, the system comprising:
a frame comprising at least two vertical bars interconnecting at least two horizontal bars, the bars being pivotable about a hinge;
a mobility mechanism disposed at a bottom end of the vertical bars;
a laminar flow subassembly supported by the horizontal bars, the laminar flow subassembly comprising one or more inlet ports having a positive pressure port, at least one tube, one or more outlet ports having a negative pressure port, and a vent;
a high-efficiency particulate air filter disposed at one or both of the vacuum ports;
an ultraviolet light disposed at one or both of the vacuum ports;
a vacuum pump operatively connected to the laminar flow subassembly, the vacuum subassembly operable to displace a gas from the positive pressure port to the negative pressure port; and
a hood substantially covering the frame, the hood being resilient and at least partially transparent, the hood being operable to substantially prevent escape of the gas from the approximate area of the frame, the hood forming at least one window and a pair of arm passageways.

19. The system of claim 18, wherein the frame is operable in proximity to a head region of a medical chair, whereby the frame is fastenable to the head region of the medical chair, or movable over the head region of the medical chair.

20. A medical chair protective barrier and gas distribution system, the system comprising:
a frame comprising four vertical bars interconnecting five horizontal bars, the bars being pivotable about a hinge, the frame being operable in proximity to a head region of a dental chair, whereby the frame is fastenable to the head region of the dental chair, or movable over the head region of the dental chair;
a mobility mechanism disposed at a bottom end of the vertical bars;
a laminar flow subassembly supported by the horizontal bars, the laminar flow subassembly comprising one or more inlet ports having a positive pressure port, at least one tube, one or more outlet ports having a negative pressure port, and a vent;
a vacuum pump operatively connected to the laminar flow subassembly, the vacuum subassembly operable to displace a gas from the positive pressure port to the negative pressure port; and
a hood substantially covering the frame, the hood being resilient and at least partially transparent, the hood being fabricated from a clear acrylic material, the hood being operable to substantially prevent escape of the gas from the approximate area of the frame, the hood forming a window, the window enabling access to the area in proximity to the frame, the hood further forming a pair of arm passageways sized and dimensioned to enable passage of a pair of arms.
